(19) United States
(12) Patent Application Publication
(10) Pub. No.: US 2016/0051692 A1
(43) Pub. Date: Feb. 25, 2016

(54) NOVEL METHODS OF USE OF BIOMIMETIC PROTEOGLYCANS

(71) Applicant: DREXEL UNIVERSITY, Philadelphia, PA (US)

(72) Inventors: Michele MARCOLONGO, Aston, PA (US); Owen C. MONTGOMERY, Sewell, NJ (US); Katsiaryna PRUDNIKOVA, Huntingdon Valley, PA (US)

(73) Assignee: DREXEL UNIVERSITY, Philadelphia, PA (US)

(21) Appl. No.: 14/784,525

(22) PCT Filed: Apr. 17, 2014
(86) PCT No.: PCT/US14/34470

§ 371 Date: Oct. 14, 2015

Related U.S. Application Data
(60) Provisional application No. 61/813,434, filed on Apr. 18, 2013.

(51) Int. Cl. A61K 47/48; A61K 9/00; A61K 38/17

(52) U.S. Cl. CPC A61K 47/48176; A61K 38/1732; A61K 9/0019

(57) ABSTRACT

In one aspect, the present invention relates to a new method of treating or preventing urinary incontinence in a mammal in need thereof. In certain embodiments, the method comprises contacting a composition comprising at least one biomimetic proteoglycan with the urethral or periurethral tissue of the mammal.

### CROSS-REFERENCE TO RELATED APPLICATIONS

The present application claims priority under 35 U.S.C. §119(e) to U.S. Provisional Application No. 61/813,434, filed Apr. 18, 2013, which application is hereby incorporated by reference in its entirety herein.

### BACKGROUND OF THE INVENTION

Urinary incontinence is the involuntary leakage of urine through the urethra. It can happen to both men and women, although women are twice as likely as men to develop it due to pregnancy, childbirth, and menopause (Brown, et al., 1999, Obstetrics and Gynecology 91(1), 66-70). It is one of the most common reasons for hospitalization, and the total estimated treatment cost in U.S. is $19.5 billion (Smith, et al., 2006, CMAJ 175(10):1233-40).

Strategies to treat urinary incontinence include surgical and non-surgical methods. Surgical procedures including slings, neurostimulators and artificial sphincters are complex and expensive, and do not work for every patient. Further, surgical intervention comes with the risk of infection, anesthesia and trauma. As an alternative to the surgical procedure, a bulking agent may be injected to the urethral or periurethral tissue for the purpose of compressing the urethra, thus blocking the involuntary flow of urine through urethra. Many bulking agents have been used, including sodium morrhuate (Murless, et al., 1938, J. Obstet. Gynaecol. 45:67-73); paraffin (Quackels, et al., 1955, Acta Urol. Belg. 23:259-262); polytetrafluoroethylene particles (Schulman, et al., 1984, BMJ 228:192); and glutaraldehyde cross-linked bovine dermal collagen (Malizia, et al., 1984, JAMA 251:3227-3281). Those bulking agents known in the art share one common problem: migration out of periurethral tissue after a short time. Consequentially, their efficacy time is short, and repeated injections into the urethra are needed.

Aggrecan, a protein modified with large carbohydrates, is the major proteoglycan in cartilage (up to 10% of its dry weight). Many individual monomers of aggrecan bind to hyaluronic acid to form an aggregate comprising up to 100 monomers attached to a single chain of hyaluronic acid (HA) (Kubo M. et al., 2009, Life Sci. 85:477-83).

An aggrecan monomer has a central protein backbone of about 210-250 kDa to which are attached both chondroitin sulfate and keratan sulfate chains. Chondroitin sulfate chains (100-150 per monomer) are located mostly in the C-terminal, while the keratan sulfate chains (30-60 per monomer) are preferentially located close to the N-terminus The hydrated structure of aggrecan provides intervertebral disc and cartilage with the ability to resist to compressive loads.

However, aggrecan itself is also susceptible to proteolytic cleavage. The interglobular domain (IGD) region is particularly susceptible to proteolytic cleavage (Flannery et al., Matrix Biol. 1998, 16, 507). The fragments after cleavage are extruded and spread into the surrounding area, contributing to the production of pro-inflammatory cytokines and matrix metalloproteases. Cytokines and matrix metalloproteases have been associated with the degradation of collagen type II fibers in cartilage (Billinghurst, et al., 1997, J. Clin. Invest. 99:1534).

Therefore there is a need in the art for novel methods of treating urinary incontinence with long lasting effect, high efficacy, and minimum side effect. The present invention fulfills this unmet need.

### BRIEF SUMMARY OF THE INVENTION

In one aspect, the present invention relates to a novel method of improving or preventing degradation of mechanical properties of a tissue in need thereof. In another aspect, the present invention relates to a novel method of treating urinary incontinence, wherein the method is characterized by long lasting effects, high efficacy and minimum side effects.

In certain embodiments, the method of the invention comprises contacting a composition comprising at least one biomimetic proteoglycan with the periurethral and/or urethral tissue of a human in need thereof. In other embodiments, the biomimetic proteoglycan functions as an effective bulking agent. In yet other embodiments, the biomimetic proteoglycan interacts with the periurethral and/or urethral matrix collagen fibrils. Such interaction enables the biomimetic proteoglycan to remain in the tissue and extends the efficacy time for the treatment. This integration offers a major advantage over the prior art bulking agents, which migrate out of the periurethral and/or urethral tissue, rendering the prior treatment clinically ineffective after about six to twelve months.

In one aspect, the invention provides a method of treating or preventing urinary incontinence in a mammal in need thereof, the method comprising contacting a composition comprising at least one biomimetic proteoglycan with the periurethral and/or urethral tissue of the mammal.

In another aspect, the invention provides a kit comprising a composition comprising at least one biomimetic proteoglycan, an applicator, and an instructional material for use thereof, wherein the instructional material comprises instructions for (i) treating or preventing urinary incontinence in a mammal and/or (ii) improving or preventing degradation of mechanical properties of a tissue.

In yet another aspect, the invention provides a method of improving or preventing degradation of mechanical properties of a tissue in need thereof, the method comprising contacting the tissue with at least one biomimetic proteoglycan.

In yet another aspect, the invention provides a method of promoting degradation of a biomimetic proteoglycan present in a tissue, the method comprising contacting at least one exogenous enzyme with the tissue, whereby the exogenous enzyme promotes at least partial breakdown of the biomimetic proteoglycan.

In certain embodiments, the step of contacting the tissue with at least one biomimetic proteoglycan comprises injecting the at least one biomimetic proteoglycan into the tissue or in the vicinity thereof.

In certain embodiments, the biomimetic proteoglycan comprises a biomimetic aggrecan. In other embodiments, the tissue comprises periurethral and/or urethral tissue.

In certain embodiments, the biomimetic proteoglycan comprises a glycosaminoglycan (GAG) that is attached to a core structure. In other embodiments, the GAG is selected from the group consisting of hyaluronic acid, chondroitin, chondroitin sulfate, heparin, heparin sulfate, dermatan sulfate, laminin, keratan sulfate, chitin, chitosan, acetyl-glucosamine, oligosaccharides, and any combinations thereof. In yet other embodiments, the core structure is selected from the group consisting of a synthetic polymer, a protein, a peptide, a nucleic acid, a carbohydrate and any combinations thereof. In yet other embodiments, the core structure is a biocompatible polymer. In yet other embodiments, the core structure is a synthetic polymer selected from the group consisting poly(4-vinylphenyl boronic acid), poly (3,3′-diethoxypropyl methacrylate), polyacrolein, poly(N-isopropyl acrylamide-co-glycidyl methacrylate), poly(allyl glycidyl ether), poly(ethylene glycol), poly(acrylic acid), epoxides and any combinations thereof.

In certain embodiments, the biomimetic proteoglycan is resistant to enzymatic breakdown in a mammalian in vivo environment. In other embodiments, at least a portion of said biomimetic proteoglycans are susceptible to enzymatic in vivo breakdown.

In certain embodiments, the GAG is attached to the core structure through a linkage selected from the group consisting of a boronic acid-diol linkage, epoxide-amine linkage, aldehyde-amine linkage, carboxylic acid-amine linkage, sulfhydryl-maleimide linkage, and any combinations thereof.

In certain embodiments, the biomimetic proteoglycan has a shape selected from the group consisting of cyclic, linear, branched, star-shaped, comb, graft, bottlebrush, dendritic, mushroom, and any combinations thereof.

In certain embodiments, the GAG comprises a terminal handle selected from the group consisting of a primary amine, diol, aldehyde, and any combinations thereof. In other embodiments, the GAG comprises chondroitin sulfate, and the core structure comprises poly(acrylic acid). In yet other embodiments, the GAG comprises keratin sulfate, and the core structure comprises poly(acrylic acid). In yet other embodiments, the GAG comprises at least one selected from the group consisting of chondroitin sulfate or keratin sulfate, and the core structure comprises poly(acrylic acid).

In certain embodiments, contacting the composition with the tissue improves or prevents further degradation of mechanical properties of the tissue. In other embodiments, as the urethral and/or periurethral tissue degenerates, there may be a stiffening of the periurethral and/or urethral tissue, which limits the closure. In certain embodiments, the methods of the invention reverse or prevent progression of the stiffening of the periurethral and/or urethral tissue. In other embodiments, a given period of time after the composition is contacted with the tissue, at least one exogenous enzyme is contacted with the tissue, whereby the exogenous enzyme promotes at least partial breakdown of the biomimetic proteoglycan.

In certain embodiments, the biomimetic proteoglycan is crosslinked to itself and/or an additional molecule to form a hydrogel. In certain embodiments, the additional molecule comprises at least one selected from the group consisting of collagen, pectin, carrageenan, poly(L-lysine), gelatin, agarose, dextran sulfate, heparin, polygalacturonic acid, mucin, chondroitin sulfate, hyaluronic acid, chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-poly(ethylene glycol) (PAA-g-PEG), poly(L-glutamic acid)-grafted-poly(ethylene glycol) (PGA-g-PEG), poly(sodium 4-styrenesulfonate) (PSS), dermatan sulfate, carboxymethyl cellulose (CMC), and any combinations thereof.

In certain embodiments, the exogenous enzyme comprises chondroitinase, and the biomimetic proteoglycan comprises chondroitin sulfate. In other embodiments, the tisuue is in vivo.

In certain embodiments, the mammal is human.

### BRIEF DESCRIPTION OF THE DRAWINGS

The following detailed description of specific embodiments of the invention will be better understood when read in conjunction with the appended drawings. For the purpose of illustrating the invention, illustrated in the drawings are specific embodiments. It should be understood, however, that the invention is not limited to the precise arrangements and instrumentalities of the embodiments illustrated in the drawings.

FIG. 1, comprising FIGS. 1(a) and 1(b), illustrates the structure of cartilage tissue (FIG. 1(a)) and structure of aggrecan (FIG. 1(a)).

FIG. 2 illustrates the structure of an aggrecan monomer.

FIG. 3 illustrates the interaction between biomimetic aggrecan and the tissue matrix.

FIG. 4 illustrates non-limiting steps for synthesis biomimetic aggrecan with polyacrylic acid (PAA) as the backbone and chondroitin sulfate (CS) as the bristles.

FIG. 5 illustrates the ¹H-NMR spectra of CS, PAA250−CS and PAA250+CS. Wherein PAA250 refers to PAA with molecular weight at 250,000 Da; PAA250−CS refers CS was covalently attached to PAA backbone; and PAA250+CS refers to a mixture of PAA250 and CS without attachment.

FIG. 6, comprising FIGS. 6(a) and 6(b), illustrates the measurement the stiffness and volume change of the tissue before injection of biomimetic aggrecan (FIG. 6(a)) and after (FIG. 6(b)).

FIG. 7 illustrates the volume change after the tissue was injected with BA. The left picture is the tissue before injection and the right picture is after the injection.

FIG. 8 illustrates infiltration of biomimetic aggrecan through periurethral and/or urethral tissue.

FIG. 9 illustrates binding studies using a BioSensor chip.

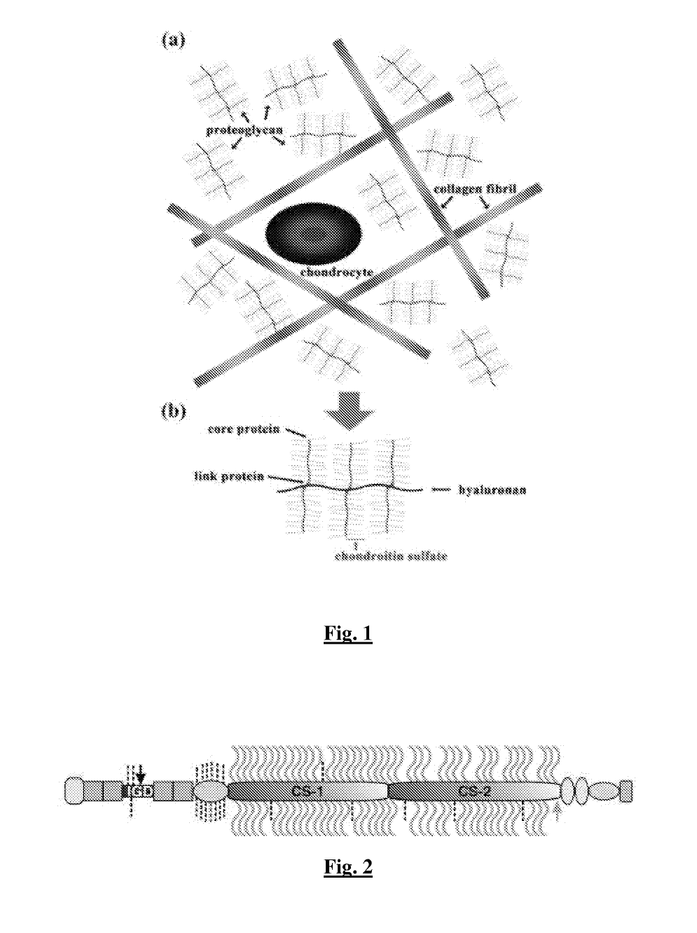

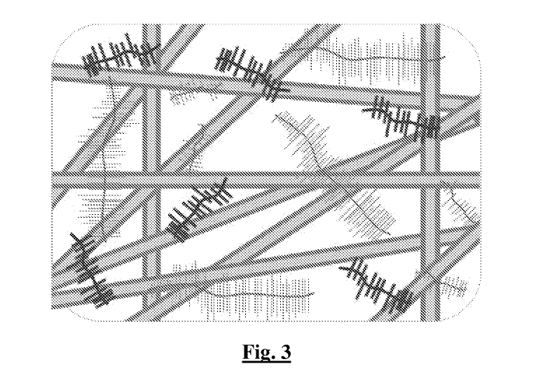

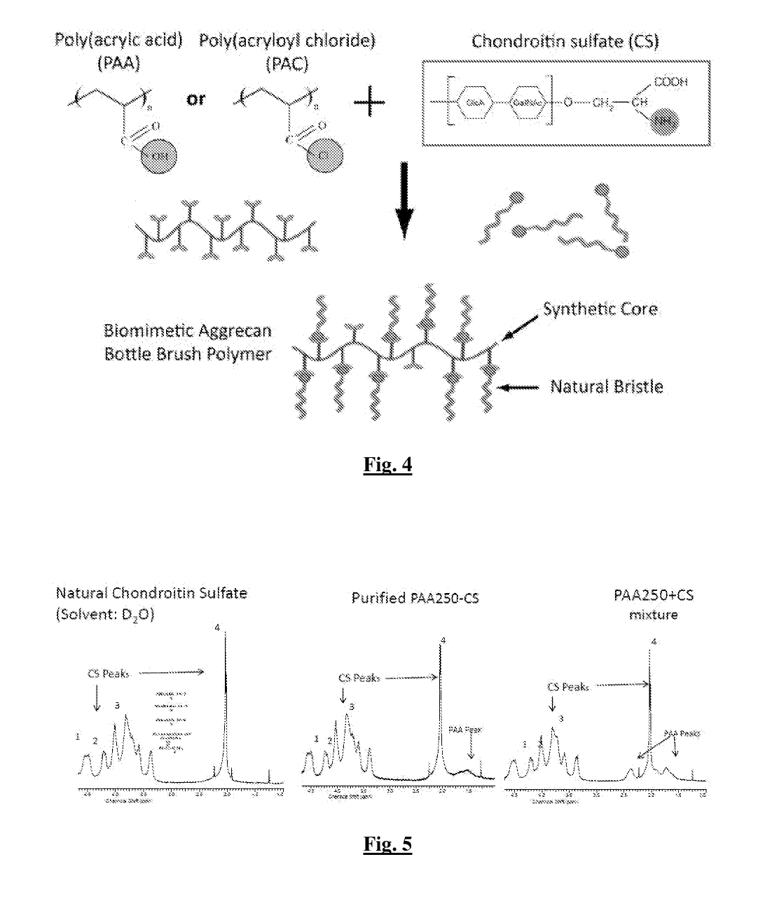

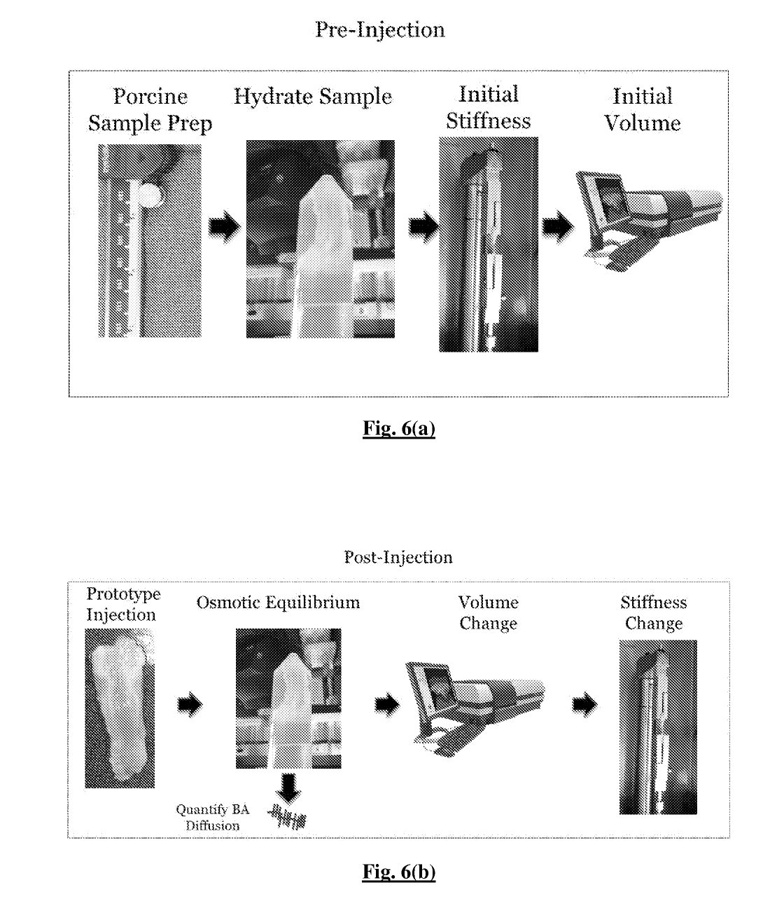

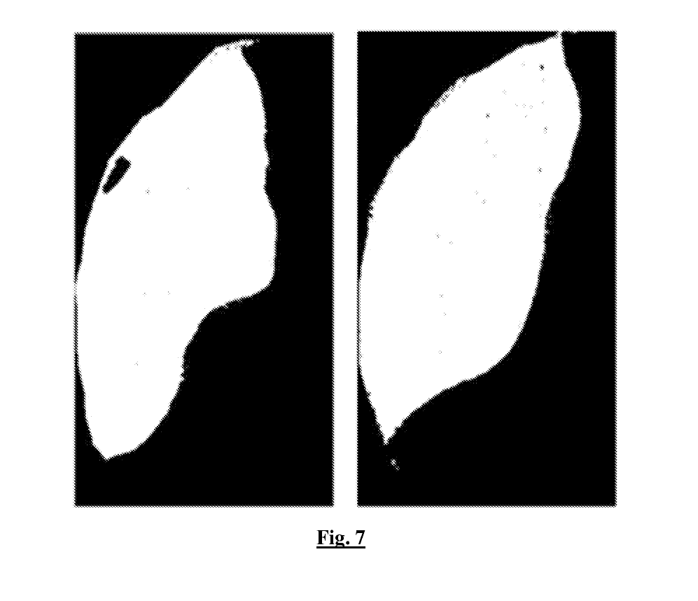

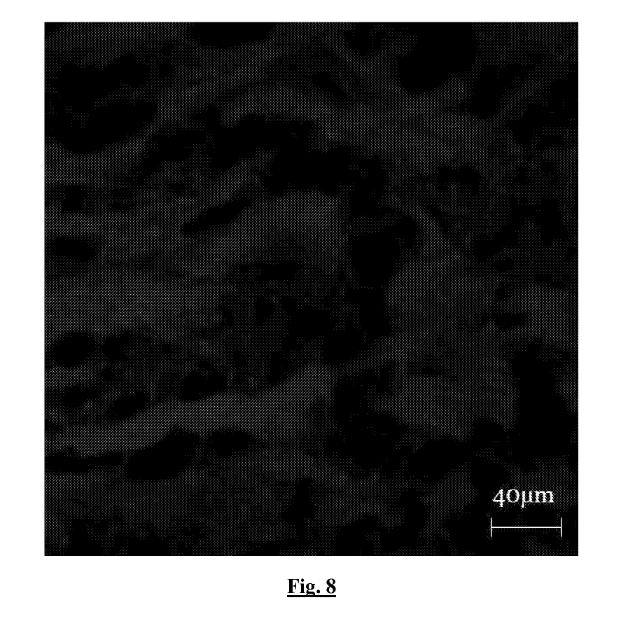

### DETAILED DESCRIPTION OF THE INVENTION

The present invention relates to a new method of treating urinary incontinence for a human in need thereof. The new method comprises contacting a composition comprising at least one biomimetic proteoglycan with the periurethral and/or urethral tissue of a human. In one aspect, the biomimetic proteoglycan in the composition functions as an effective bulking agent. In another aspect, the biomimetic proteoglycan interacts with the periurethral and/or urethral matrix collagen fibrils; such interaction enables the biomimetic proteoglycan to remain in the tissue and extends the efficacy time for the treatment. The method of the invention is advantageous over prior art methods that used bulking agents shown to migrate out of the urethral and/or periurethral tissue, rendering those treatment clinically ineffective after about six months.

In certain embodiments, once contacted with the tissue, the biomimetic proteoglycan mimics the chondroitin sulfate proteoglycan aggrecan at least in terms of hydration, structure support, and interaction with the periurethral and/or urethral matrix collagen fibrils. Without wishing to be limited by any theory, the biomimetic proteoglycans can attract water molecules from its vicinity due to its high osmotic potential created by the high density of negative charge in GAG moiety. This process leads to the volume increase (bulking) and stiffness decrease of the tissue. Also, the three-dimensional architecture of the biomimetic proteoglycans provides structural support to the surrounding tissue.

In certain embodiments, the biomimetic proteoglycan integrates with the existing collagen in the extracellular matrix through its chondroitin sulfate bristles. In other embodiments, the chondroitin sulfate bristles bind to collagen. In yet other embodiments, the biomimetic proteoglycan modulates the properties of the matrix by virtue of specifically interacting with molecules of the surrounding tissue. In yet other embodiments, the biomimetic proteoglycan agglomerates and results in tissue augmentation.

In certain embodiments, the biomimetic proteoglycan deliver a drug to the tissue.

In certain embodiments, the biomimetic proteoglycan forms a gel with itself and/or an additional biomolecule or polymer. In other embodiments, the gel bulks the tissue. In yet other embodiments, the biomimetic proteoglycan forms a gel, which delivers drugs to the tissue.

In certain embodiments, the biomimetic proteoglycan stimulates cell regeneration in the tissue. In other embodiments, the combination of a drug and the biomimetic proteoglycan stimulates cell regeneration in the tissue. In yet other embodiments, the biomimetic proteoglycans reduces inflammation in the tissue.

In certain embodiments, the biomimetic proteoglycans are prepared by attaching glycosaminoglycan (GAG) to a biocompatible polymer to minimize potential side effects, such as but not limited to tissue rejection, and allergic response. Biocompatible polymers are well known in the art and include, by example, cellulose acetates, ethylene vinyl alcohol copolymers, polyalkylacrylates, polyacrylonitrile, polyacrylic acid and the like.

### Definitions:

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “aggrecan” refers to the aggrecan monomer.

As used herein, the term “aggregated aggrecan” refers to aggrecan that is linked to hyaluronic acid in an end-on configuration and comprises about 100 aggrecan monomers.

As used herein, a disease or disorder is “alleviated” if the severity of a symptom of the disease or disorder, the frequency with which such a symptom is experienced by a patient, or both, is reduced.

The term “biocompatible polymer” refers to a polymer which is non-toxic, chemically inert, non-irritable, insoluble and substantially non-immunogenic when contacted with human tissue.

The term “biologically compatible” or “biocompatible” refers to reagents, cells, compounds, materials, compositions, and/or dosage formulations that are suitable for use in contact with the tissues of human beings and animals without excessive toxicity, irritation, allergic response, or other complication commensurate with a reasonable benefit/risk ratio.

As used herein, the term “core structure” refers to the moiety or structure for which GAGs, such as chondroitin sulfate, can attach to form a biomimetic proteoglycan. In some instances, the core structure is considered the polymer backbone, core portion, polymer core, or protein portion of the proteoglycan. In some instances, the core structure can be a synthetic polymer, protein, peptide, nucleic acid, carbohydrate or combinations thereof.

As used herein, the term “Da” refers to the unified atomic mass unit or Dalton, which is the standard unit of indicating mass of an atom or a molecular.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “glycosaminoglycan” and “GAG”, as used interchangeably herein, refer to a macromolecule comprised of carbohydrate. The GAGs for use in the present invention may vary in size and be either sulfated or non-sulfated. The GAGs which may be used in the methods of the invention include, but are not limited to, hyaluronic acid, chondroitin, chondroitin sulfates (e.g., chondroitin 6-sulfate and chondroitin 4-sulfate), heparin, heparin sulfate, dermatan sulfate, laminin, keratan sulfate, chitin, chitosan, acetyl-glucosamine, and the like.

As used herein, “grafting from” refers to a process based on the synthesis of a macroinitiator containing suitable initiating groups along the backbone. The high initiator efficiency, the ability to manipulate initiator distribution along the main chain and the side chain length control afforded by living polymerization techniques makes the “grafting from” process an attractive option in the synthesis of well-defined graft copolymers.

As used herein, “grafting through” refers to a process of the synthesis of a well-defined macromonomer, followed by its copolymerization with a low molecular weight comonomer. Control over length and polydispersity can be achieved for both backbone and side chains using this methodology. The approach is characteristic of a multistep synthesis and the grafting density is associated with the reactivity ratios of the macromonomers.

As used herein, the term “grafting to” or “grafting onto” refers to end-functionalized polymer chains that are attached to the main chain of another polymer through coupling reactions with one or more functional groups along its backbone.

As used herein, an “instructional material” includes a publication, a recording, a diagram, or any other medium of expression that can be used to communicate the usefulness of a composition or method of the invention in the kit for treating, preventing or alleviating various diseases or disorders recited herein. The instructional material of the kit of the invention can, for example, be affixed to a container that contains the identified composition or delivery system of the invention or be shipped together with a container that contains the identified composition or delivery system. Alternatively, the instructional material can be shipped separately from the container with the intention that the instructional material and the composition be used cooperatively by the recipient.

As used herein, the term “biomimetic aggrecan” refers to a material that mimics at least certain aspects of the natural structure and function of natural aggrecan, such as, but not limited to, hydration, structure support, and interaction with the periurethral and/or urethral matrix collagen fibrils.

As used herein, the term “biomimetic proteoglycan” refers to a material that mimics at least certain aspects of the natural structure and function of natural proteoglycan, such as, but not limited to, hydration, structure support, and interaction with the periurethral and/or urethral matrix collagen fibrils.

As used herein, the terms “patient,” “subject,” “individual” and the like are used interchangeably, and refer to any animal, or organs, tissues or cells thereof whether in vitro or in situ, amenable to the methods described herein. In certain embodiments, the patient, subject or individual is a vertebrate. In other embodiments, the patient, subject or individual is a mammal. In yet other embodiments, the patient, subject or individual is a human.

As used herein, the term “periurethral tissue” refers to the tissue surrounding the urethra in a mammal.

As used herein, the term “urethral tissue” refers to the tissue that is part of the urethra in a mammal

As used herein, the term “endogenous enzyme” refers to the enzyme that originates from the particular tissue of a subject.

As used herein, the term “exogenous enzyme” refers to the enzyme that does not originate from the particular tissue of a subject.

As used herein, “terminal handle” refers to the functional group at the end of GAG, through which the GAG is attached to the core structure.

As used herein, the term “treating a disease or disorder” means reducing the frequency with which a symptom of the disease or disorder is experienced by a patient. Disease and disorder are used interchangeably herein.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 and the like, as well as individual numbers within that range, for example, 1, 2, 2.7, 3, 4, 5, 5.3, and 6. This applies regardless of the breadth of the range.

### Disclosure

In one aspect, biomimetic proteoglycans mimic the structure and functionality of natural proteoglycans, and in other aspects it can be designed and prepared to resist enzymatic cleavage. It may be prepared by attaching a GAG chain via a covalent bond to a core structure. The covalent bond can be formed through, for example, boronic acid-diol reaction, epoxide-amine reaction, aldehyde-amine reaction, carboxylic acid-amine reaction, sulthydryl-maleimide reaction, and any combinations thereof. Compositions comprising biomimetic proteoglycan s are described in U.S. Application Publication No. US 2013/0052155 A1, which is hereby incorporated herein by reference in its entirety.

In certain embodiments, the biomimetic proteoglycans can be prepared by attaching a terminal diol in chondroitin sulfate to a boronic acid-functionalized polymer. Utilizing the high affinity complexation of boronic acids with compounds containing diols (such as saccharides), novel biomimetic proteoglycans may be prepared, wherein the attached chondroitin sulfate chains form brush “bristles” to mimic the bristles of the aggrecan molecule.

In other embodiments, the biomimetic proteoglycans can be prepared by attaching at least chondroitin sulfate through a terminal primary amine handle to a diverse array of polymer backbones. In another embodiment, a primary amine interaction is available only in the terminal region of the chondroitin sulfate molecule. This allows for the controlled organization of chondroitin sulfate onto various polymeric backbones that may be tuned to match the properties desired for treating urinary incontinence.

In yet other embodiments, the biomimetic proteoglycans can be prepared using a grafting method wherein chondroitin sulfate or other similar GAG chain is grafted to a functional polymer. The functional polymer can be, but is not limited to, any polymer with groups that may react with diols or primary amines, such as boronic acids, epoxides, aldehydes and/or carboxylic acids. An example of a possible “grafted to” polymer is poly(acrylic acid), which is a carboxylic acid-containing linear polymer. Poly(acrylic acid) may be activated with 1-ethyl-3-(3-dimethylaminopropyl)carbodiimide hydrochloride (EDC)/N-hydroxysulfosuccinimide (NHS) and then reacted with the terminal primary amine of chondroitin sulfate thus creating a bottle brush structure.

In yet other embodiments, the biomimetic proteoglycans are prepared through the “grafting through” method, wherein the chondroitin sulfate or other similar GAG chain is modified with a polymerizable end group which is subsequently homo- or co-polymerized to form a bottle brush polymer. As an example of a possible “grafted through” polymer, 2-vinyloxirane may be attached to chondroitin sulfate through an interaction of the terminal primary amine in chondroitin sulfate with the epoxide of 2-vinyloxirane, thus creating a vinyl chondroitin sulfate. The vinyl chondroitin sulfate may be subsequently polymerized via free radical polymerization. Similarly, another example is the attachment of poly(4-vinylbenzylboronic acid) to chondroitin sulfate via an interaction of the terminal diol in chondroitin sulfate with the boronic acid in poly(4-vinylbenzylboronic acid) to form a boronic ester. The vinyl-containing chondroitin sulfate is then subsequently polymerized via free radical polymerization to form a bottle brush polymer.

In yet other embodiments, the biomimetic proteoglycans are prepared through the grafting method wherein a disaccharide unit of chondroitin sulfate (GlcUA and GalNAc) or other GAG is attached to a polymeric backbone, through but not limited to aldehyde or amine interactions. Subsequent disaccharide or saccharide units are then grown from the polymeric backbone using enzymes of GAG synthesis, such as but not limited to GlcA I transferase, GlaNAc transferase, chondroitin synthase, chondroitin 6-sulfotransferase and chondroitin 4-O-sulfotransferase.

In yet other embodiments, the biomimetic proteoglycans prepared through any of the grafting methods are end-functionalized with, but not limited to, a hyaluronic binding region or collagen binding region. Polymerizations that can be utilized to incorporate a functional group on the terminal end of the biomimetic proteoglycans bottle brush include, but are not limited to, radical polymerization, cationic polymerization, living anionic polymerization, atom transfer radical polymerization, and ring opening metathesis polymerization.

Based on the disclosure herein, a skilled artisan would understand that the biomimetic proteoglycans can be engineered to encompass any type of glycosaminoglycan and combinations thereof with any type of core protein or polymer core.

Although natural aggrecan monomer exists in bottlebrush shape, it is understood that biomimetic proteoglycans, by design, may have a shape selected from the group consisting of cyclic, linear, branched, star-shaped, comb, graft, bottlebrush, dendritic, mushroom, and any combinations thereof.

In certain embodiments, when biomimetic proteoglycans are arranged in a bottle-brush structure, such that the electrostatically charged bristle molecules are in close proximity to one another, the charged bristles provide electrostatic repulsions and steric hindrances that assist the biomimetic proteoglycans in resisting force. This allows for an increased osmotic potential as well as improved mechanical function. Higher osmotic potential leads to greater retention of water by the polymer from the surroundings and stronger bulking effect.

In certain embodiments, the density of the bristles in the biomimetic proteoglycans is controllable, based on the careful selection of starting materials for the preparation of the biomimetic proteoglycans, whereby the spacing among the bristles can be varied to afford compositions of desired properties. In other embodiments, the bristle density is such that spacing among the bristles ranges from about 2 nm to about 5 nm. In yet other embodiments, the bristle density is such that spacing among the bristles is equal to or less than about 2 nm In yet other embodiments, the bristle density is such that spacing among the bristles is equal to or greater than 5 nm.

It will be understood that all kinds of glycosaminoglycans can be used to form a biomimetic proteoglycan. For example, suitable glycosaminoglycans include HA, chondroitin, chondroitin sulfate, dermatan sulfate, heparan sulfate, keratin sulfate and heparin. In addition, any polymer that resembles a glycosaminoglycan can be used to generate the biomimetic proteoglycans. Based on the disclosure presented herein, a skilled artisan would understand that any hydrophilic polymer can be used within the compositions of the invention.

In certain embodiments, the core structure is selected from the group consisting of a synthetic polymer, a protein, a peptide, a nucleic acid, a carbohydrate and any combinations thereof. In other embodiments, the core structure is a synthetic polymer selected from the group consisting poly(4-vinylphenyl boronic acid), poly(3,3′-diethoxypropyl methacrylate), polyacrolein, poly(N-isopropyl acrylamide-co-glycidyl methacrylate), poly(allyl glycidyl ether), poly(ethylene glycol), poly(acrylic acid), epoxides, and any combinations thereof. In certain embodiments, the biomimetic proteoglycans comprises a bottle brush-like molecule with chondroitin sulfate (CS) bristles and polyacrylic acid as the polymer backbone. In other embodiments, the size of the biomimetic proteoglycans can be controlled so that a desired size is generated.

In certain embodiments, the biomimetic proteoglycans are resistant to endogenous enzymatic digestion, so that the composition does not undergo enzymatic breakdown over a period of time, allowing for the repeated introduction of distinct components of the biomimetic proteoglycans onto an existing structure. Therefore, a large macromolecular sized biomimetic proteoglycans can be generated over time. In addition to the ability to generate desired sizes of biomimetic proteoglycans, it is also possible to generate biomimetic proteoglycans that are variably susceptible to enzymatic digestion. In certain embodiments, at least a portion of the biomimetic proteoglycans is susceptible to either endogenous or exogenous enzymatic digestion. Specific breakdown of the molecule by exogenous enzymes may be used as a method to reverse the effects of iintroducing biomimetic proteoglycans into the subject. In one embodiment, the chondroitin sulfate bristles are degraded using exogenous chondroitinase, such as bacterially derived chondritinase, such as chondroitinase ABC.

In certain embodiments, the biomimetic proteoglycans are injectable by a delivery device or can be formulated to be injectable.

The invention further provides a kit for treating urinary incontinence. In certain embodiments, the kit comprises a composition, an applicator, and a delivery device, wherein the composition comprises at least one biomimetic proteoglycan.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Synthesis of Biomimetic Aggrecan

Synthesis of biomimetic aggrecan has been described in the art (Sarkar, et al, 2012, Carbohydrate Polymers 90 (1):431-440). For illustration purpose only, the terminal portion of CS is functionalized and the resulting group is reacted with an appropriate group on the core structure to form a covalent bond. This kind of chemistry link used for such attachment may comprise, for example, amine-epoxy, diol-boronic acid and so forth. show the diagram of synthesis biomimetic aggrecan by either “grafting to” or “grafting through” technology.

Synthesis of Biomimetic Aggrecan with Polyacrylic Acid (PAA) as Polymer Backbone

PAA of 250kDa MW, 1 OOkDa MWwere purchased from Sigma.

For reaction with CS, PAA (33 mg/mL, 0.132 mM PAA) was first activated with EDC/sulfo-NHS (2 mM EDC in DMSO and 5 mM sulfo-NHS in MES buffer (0.05M MES, 0.5M NaCl), pH 6.0) for 15-20 min followed by filtering using a Sephadex G-25 pre-packed desalting column (PD-10, GE Healthcare) to remove excess reactants. The column was centrifuged at 1500 G for 2 minutes. Activated PAA was then combined with a CS solution prepared in PBS. Solution pH was adjusted with NaOH to pH 7.4 and the mixture was allowed to react over time mixed continuously (Thermolyne Labquake).

After 72 hours of reaction, samples were dialyzed in 50,000 Molecular Weight Cut Off (MWCO) membranes (Spectrum Labs, Rancho Dominguez, Calif.) against DI water for 96 hours, then frozen and lyophilized Purified samples were reconstituted and used for further characterization.

### Example 2

### Characterization of Biomimetic Aggrecan with PAA as the Backbone

### ¹H-NMR

1 mg/ml lyophilized samples were solubilized in deuterium oxide (D₂O). ¹H-NMR spectra were taken on a 300 MHz NMR spectrometer (UNITYNOVA, Mckinley Scientific, Sparta, N.J.) at 64 scans and at ambient temperature.

Enzymatic Stability of Biomimetic Aggrecan with PAA as the Polymer Backbone

The enzymatic stabilities of biomimetic aggrecan and its major components (the synthetic polymer core and CS side-chains) were evaluated. These studies were observed using Blyscan, DMMB (1,9-dimethylmethylene blue).

CS bristle degradation can be analyzed chemically through glycosaminoglycan (GAG) content assays, using either 1,9-dimethylmethylene blue (DMMB) (Melrose et al., 2001, Journal of Anatomy, 198:3-15) or pre-prepared Blyscan (Biocolor) kits. First, the reliability of the assays was confirmed by preparing a CS standard curve. The enzyme studies were designed with a positive and negative control. The enzyme degradation samples were heated to a biological temperature of 37° C. The change in absorption of samples with and without enzyme was an indication of GAG content, and therefore extent of degradation. These assays were analyzed using chondroitinases, hyaluronidase, and both enzymes simultaneously for comparison.

### Isolation of Glycosaminoglycan-1,9-Dimethylmethylene Blue (GAG-DMMB) Complex for Standard Curve

A formate buffer was prepared using 5% (v/v) ethanol, 0.2 M guanidine hydrochloride, sodium formate and 98% (v/v) formic acid for a final pH of 3.0. A decomplexation solution was prepared using 4 M of guanidine hydrochloride in a 50 mM sodium acetate buffer (pH 6.8). The 10% (v/v) propan-1-ol may be added for increased solution stability. DMMB was dissolved in ethanol and filtered through filter paper. AIM guanidine hydrochloride solution with sodium formate and 98% (v/v) formic acid was mixed with the DMMB ethanol solution. The final volume was complete to 500 ml and immediately diluted (1:1) with the formate buffer.

### DMMB Assay for CS Standard Curve

The detectable range for DMMB is 0.5-5 um of glycosaminoglycan. Solutions were serially diluted from 4 μg-0.5 μg, a total volume of 100 μl was used for each sample type. Two tubes were prepared per sample type, and three aliquots from each tube were tested for absorbance. 1× PBS was used as a negative control, both processed with DMMB and non-processed. 1 ml of DMMB solution was added to 100 μl samples and vigorously vortexed for 30 minutes. Samples were centrifuged at 13,500 G for 10 minutes. The supernatant was removed, however the pellet was saved. The decomplexation agent (1 ml/sample) was used to dissolve the pellet completely and samples were shaken for 30 minutes. Samples were plated at 200 μl/well and absorbance was read at 570 nm. Blyscan samples were read at 656 nm

### Enzyme Preparation

Mammalian hyaluronidase (Sigma) was equilibrated to room temperature. A concentrated solution was dissolved in a Tris/Acetate/Bovine Serum Albumin (BSA) buffer and diluted to 5 Units/ml.

New bottles of Chondroitinase ABC were reconstituted in 1 ml of 0.01% (w/v) BSA. A 0.1 Unit/ml solution was used for enzymatic digestion studies.

Biomimetic aggrecan to be digested was prepared at 16 mg of lyophilized powder in 1 ml of Tris/Acetate/BSA buffer. The solution was separated into 3 tubes of 100 μl, labeled control 1, control 2, or sample. Control 1 and 2 had an additional 1 ml of buffer added; Control 1 was kept at room temperature and Control 2 was heated to 37° C. The tube labeled ‘sample’ had 1 ml of enzyme solution added.

### Example 3

### Infiltration of Biomimetic Aggrecan Through Periurethral Tissue

The interaction between biomimetic aggrecan and the periurethral and/or urethral tissue was investigated.

Biomimetic proteoglycans (250 kDa PAA core with CS bristles) were fluorescently labelled and injected into porcine urethral tissue (8 mg/mL) to investigate the interaction of biomimetic proteoglycans with the tissue. After injection, the sample was equilibrated for 2 hours, cryosectioned and imaged using confocal microscopy.

As shown in FIG. 8, the biomimetic proteoglycan was able to infiltrate and integrate with the tissue throughout the urethra. This distribution of biomimetic proteoglycans throughout the tissue (rather than as a bolus or agglomerated mass) demonstrates the molecular engineering of the tissue by injection of biomimetic proteoglycans. In certain embodiments, the molecular engineering with biomimetic proteoglycans allows for modulation of mechanical properties of the tissue after injection. This study demonstrates the molecular engineering approach to the use of biomimetic proteoglycans bulking the periurethral and/or urethral tissue as a method to reduce or completely arrest urinary incontinence.

### Example 4

### Tensile Stiffness Test

The stiffness change of periurethral and/or urethral tissue before and after injection of BA was investigated.

Porcine urethra specimens were sectioned longitudinally and mechanically tested in tension before and after injections of either PBS (phosphate buffered saline controls) or one of three different concentrations (25, 50 and 100 mg/mL PBS) of biomimetic proteoglycans (250 kDa core PAA with CS bristles). The strain rate we used in our testing procedure was 0.8 mm/s to 40% strain. Tensile testing was performed using the KES G1 Tensile Tester and LabVIEW software. Samples were permitted to equilibrate in PBS for one hour after injection and prior to testing. Samples showed reduced stiffness after injections with biomimetic proteoglycans compared to PBS control, as predicted (Table 1).

TABLE 1

| | |
|---|---|
| Concentration (mg of | Average % |
| biomimetic aggrecan | Decrease in |
| in mL of PBS) | Stiffness |

| | | |
|---|---|---|
| 25 mg/mL | (n = 3) | 40.92 |
| 50 mg/mL | (n = 3) | 49.32 |
| 100 mg/mL | (n = 3) | 30.64 |
| PBS Control | (n = 2) | 1.30 |

### Example 5

### Micro CT Test

The volume change of the periurethral and/or urethral tissue before and after injection of BA was investigated. Micro CT was performed on porcine urethral tissue as received (after hydration for one hour) and after injection with one of three different concentrations (25, 50 and 100 mg/mL PBS) of biomimetic proteoglycans (250 kDa core PAA with CS bristles) to assess the volume change before and after injecting the tissue samples with the biomimetic proteoglycan solutions. The scans were done 1 hour after the sample had been hydrated and 1 hour after the sample was injected with the BA solution. Low resolution scans were run with a camera setting of far, large pixels at 1000×540, and 34.5 μm zoom, placing the sample vertically in a 2×6 cm cylindrical canister. Images were reconstructed and analyzed through the NRecon and CTAn software offered by SkyScan™. The volume was calculated by CTAn using the white voxels, indicating solid material, from the binary selection in the volume of interest (VOI) multiplied by the voxel volume. The images were integrated into a volume model from this selection. Volume changes are shown in Table 2. An increase in volume after biomimetic proteoglycan injections was determined

TABLE 2

| | |
|---|---|
| Concentration (mg of | Average % |
| biomimetic aggrecan | Increase in |
| in mL of PBS) | Middle Volume |

| | | |
|---|---|---|
| 25 mg/mL | (n = 3) | 13.73 |
| 50 mg/mL | (n = 3) | 22.92 |
| 100 mg/mL | (n = 3) | 10.28 |
| PBS Control | (n = 2) | 8.55 |

### Example 6

### Binding Assay

A binding assay was conducted using a BioSensor chip, testing the affinity of the biomimetic aggrecan, natural aggrecan, and individual chondroitin sulfate molecules to collagen type II.

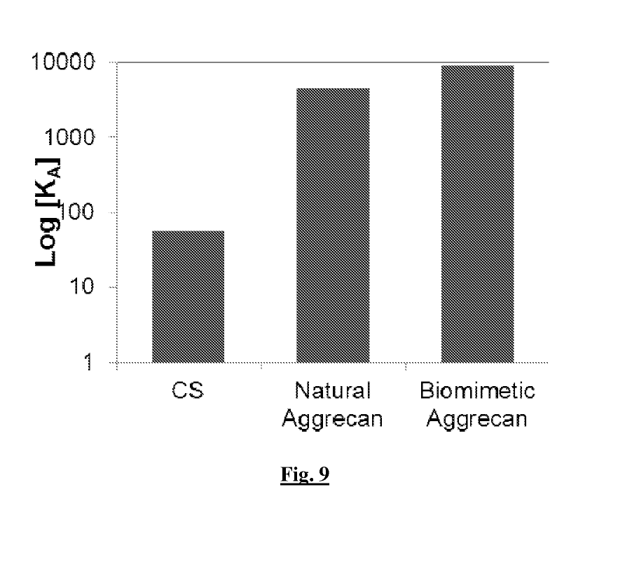

As illustrated in FIG. 9, the biosensor results indicated that natural aggrecan and biomimetic aggrecan have similar binding affinities to collagen type II, while CS binding affinity was two orders of magnitude lower, indicating the significance of the bottle-brush architecture in aggrecan/collagen interactions. The relative equivalence of biomimetic proteoglycans (250 kDa core with CS bristles and PAA core) indicated that the biomimetic proteoglycans attaches to collagen with the same affinity as the natural proteoglycan aggrecan.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing urinary incontinence in a mammal in need thereof, the method comprising contacting the urethral or periurethral tissue of the mammal with a composition comprising at least one biomimetic proteoglycan, wherein the biomimetic proteoglycan comprises at least one glycosaminoglycan (GAG) that is attached to a, core structure.

2. The method of claim 1, wherein contacting the tissue with the composition comprises injecting the composition into the tissue.

3. (canceled)

4. (canceled)

5. The method of claim 1, wherein the GAG is selected from the group consisting of hyaluronic acid, chondroitin, chondroitin sulfate, heparin, heparin sulfate, dermatan sulfate, laminin, keratin sulfate, chitin, chitosan, acetyl-glucosamine, oligosaccharides, and any combinations thereof.

6. The method of claim 1, where the core structure is selected from the group consisting of a synthetic polymer, a protein, a peptide, a nucleic acid, a. carbohydrate and any combinations thereof.

7. The method of claim 6, wherein the synthetic polymer is selected from the group consisting poly(4-vinylphenyl boronic acid), poly(3,3′-diethoxypropyl methacylate), polyacrolein, poly(N-isopropyl acrylamide-co-glycidyl methacrylate), poly(allyl glycidyl ether), poly(ethylene glycol), poly(acrylic acid), epoxides, and any combinations thereof.

8. The method of claim 1, wherein the biomimetic proteoglycan is resistant to enzymatic in vivo breakdown.

9. The method of claim 1, wherein at least a portion of the biomimetic proteoglycan is susceptible to enzymatic in vivo breakdown.

10. The method of claim 1, wherein the GAG is attached to the core structure through a linkage selected from the group consisting of a boronic acid-diol linkage, epoxide-amine linkage, aldehyde-amine linkage, carboxylic acid-amine linkage, sulthydryl-maleimide linkage, and any combinations thereof.

11. The method of claim 1, wherein the biomimetic proteoglycan has a. shape selected from the group consisting of cyclic, linear, branched, star-shaped, comb, graft, bottlebrush, dendritic, mushroom, and any combinations thereof.

12. The method of claim 1, wherein the GAG comprises a terminal handle selected from the group consisting of a primary amine, diol, aldehyde, and any combinations thereof.

13. The method of claim 1, wherein the GAG comprises at least one selected from the group consisting of chondroitin sulfate and keratin sulfate, and the core structure comprises poly(acrylic acid).

14. (canceled)

15. The method of claim 1, wherein contacting the composition with the tissue improves or prevents further degradation of mechanical properties of the tissue.

16. The method of claim 1, wherein, a given period of time after contacting the composition with the tissue, at least one exogenous enzyme is contacted with the tissue, whereby the exogenous enzyme promotes at least partial breakdown of the biomimetic proteoglycan.

17. The method of claim 1, wherein the biomimetic proteoglycan is crosslinked to itself or an additional molecule to form a hydrogel.

18. The method of claim 17, wherein the additional molecule comprises at least one selected from the group consisting of collagen, pectin, carrageenan, poly(L-lysine), gelatin, agarose, dextran sulfate, heparin, polygalacturonic acid, mucin, chondroitin sulfate, hyaluronic acid, chitosan, alginate, alginate sulfate, poly(acrylic acid), poly(methyl methacrylate (PMMA), poly(vinylsulfonic acid) (PVSA), poly(L-aspartic acid), poly(L-glutarnic acid), poly(L-aspartic acid)-grafted-poly(ethylene glycol) (PAA-g.-PEG), poly(L-glutamic acid)-grafted-poly(ethylene glycol) (PGA-g-PEG), poly(sodium 4-styrenesulfonate) (PSS), dermatan sulfate, carboxymethyl cellulose (CMC), and any combinations thereof.

19. (canceled)

20. A kit comprising a composition comprising at least one biomimetic proteoglycan, an applicator, and an instructional material for use thereof, wherein the instructional material comprises instructions for at least one selected from the group consisting of (i) treating or preventing urinary incontinence in a mammal and (ii) improving or preventing degradation of mechanical properties of a tissue in need thereof.

21. (canceled)

22. The kit of claim 20, wherein the biomimetic proteoglycan comprises at least one glycosaminoglycan (GAG) that is attached to a core structure.

23. The kit of claim 20, wherein the GAG comprises at least one selected from the group consisting of chondroitin sulfate or keratin sulfate, and the core structure comprises poly(acrylic acid).

24-37. (canceled)
